(19) United States
(12) Patent Application Publication
(10) Pub. No.: US 2017/0157429 A1
(43) Pub. Date: Jun. 8, 2017

(54) SYSTEMS, DEVICES, AND METHODS FOR PULMONARY TREATMENT

(71) Applicant: DYMEDSO, INC., Boisbriand (CA)

(72) Inventors: Manya DEEHR, Newtown, PA (US); Hugo CHARBONNEAU, Montreal (CA)

(21) Appl. No.: 15/356,001

(22) Filed: Nov. 18, 2016

Related U.S. Application Data
(60) Provisional application No. 62/257,496, filed on Nov. 19, 2015.

(51) Int. Cl. A61N 7/00; A61H 23/02

(52) U.S. Cl. CPC A61N 7/00; A61H 23/0245

(57) ABSTRACT

An apparatus includes a frequency generator to generate an electrical signal. The apparatus also includes an acoustic transducer operably coupled to the frequency generator. The acoustic transducer converts the electrical signal into a first acoustic signal. The apparatus also includes an applicator operably coupled to the acoustic transducer. The applicator includes an acoustic generator to generate a second acoustic signal based on the first acoustic signal. The applicator also includes an applicator interface configured to apply the second acoustic signal to a patient.

### CROSS-REFERENCES TO RELATED APPLICATIONS

This application claims priority to U.S. provisional application Ser. No. 62/257,496, filed Nov. 19, 2015, entitled “SYSTEMS, DEVICES, AND METHODS FOR PULMONARY TREATMENT,” the entire disclosure of which is incorporated herein by reference in its entirety.

### BACKGROUND

Cystic fibrosis (CF), chronic bronchitis, bronchiectasis, immotile cilia syndrome, asthma, and some acute respiratory tract infections can lead to abnormal airway clearance or increase sputum production. Airway secretions can be cleared by mechanisms such as mucociliary clearance (MCC), cough, peristalsis, two-phase gas-liquid flow, and alveolar clearance. The underlying pathology of abnormal airway clearance can differ from one illness to another. Chest physiotherapy (CPT) typically refers to a treatment program that attempts to compensate for abnormal airway clearance. By removing mucopurulent secretions, CPT can decrease airway obstruction and its consequences, such as atelectasis and hyperinflation. Furthermore, physiotherapy can decrease the rate of proteolytic tissue damage by removing infected secretions.

Accordingly, there is a need to provide airway clearance therapy and to promote bronchial drainage by inducing vibration in the chest walls. For example, acoustic airway treatment can induce oscillatory sound waves in the chest by means of an electro-acoustical transducer (also referred to as a “power head”), which is placed externally on the patient's chest. Power heads are typically bulky and heavy, making it challenging not only to manipulate the power head during treatment, but to treat younger patients such as infants, since the power head in conventional acoustic airway treatment is usually directly placed on a patient's chest, thereby imposing a force on the patient. Furthermore, it is usually challenging to optimize the power of acoustic waves applied to the patient. Excessive power can injure the patient, while insufficient power can decrease the treatment efficiency.

### SUMMARY

In some embodiments, an apparatus includes a signal generator configured to generate an electrical signal, and an acoustic transducer operably coupled to the signal generator. The acoustic transducer is configured to convert the electrical signal into a first acoustic signal. The apparatus also includes an applicator operably coupled to the acoustic transducer, the application configured to receive the first acoustic signal and to generate a second acoustic signal based on the first acoustic signal. The applicator is further configured to apply the second acoustic signal to a patient.

In some embodiments, a method includes generating an electrical signal using a frequency generator. The method also includes converting the electrical signal into a first acoustic signal using an acoustic transducer, and generating a second acoustic signal in an applicator based at least in part on the first acoustic signal. The method also includes applying the second acoustic signal to a patient via the applicator.

In some embodiments, an apparatus for airway treatment includes a signal generator and an acoustic transducer. The signal generator is configured to generate an electrical signal. The acoustic transducer includes a transducer membrane operably coupled to the frequency generator. The transducer membrane is configured to convert the electrical signal into a first acoustic signal. The apparatus also includes a flexible acoustic waveguide operably coupled to the acoustic transducer. The flexible acoustic waveguide is configured to guide the first acoustic signal. The apparatus further includes an applicator operably coupled to the flexible acoustic waveguide. The applicator includes an applicator membrane configured to generate a second acoustic signal based on the first acoustic signal, and an applicator interface configured to apply the second acoustic signal to a patient. The applicator membrane is recessed from the applicator interface so as to avoid contact with the patient.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the subject matter described herein. The drawings are not necessarily to scale; in some instances, various aspects of the subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is an illustration of a system for airway treatment, according to embodiments.

FIG. 2 is an illustration of an example system including an acoustic waveguide and an applicator for airway treatment, according to embodiments.

FIG. 3 is an illustration of an example system including two applicators for airway treatment, according to treatments.

FIGS. 4A and 4B are illustrations of adapters that can be used in airway treatment systems, according to embodiments.

FIG. 5 is an illustration of an example apparatus including a signal transducer, tubing, and a signal applicator, according to embodiments.

FIGS. 6A-6E are various views of an example acoustic signal applicator according to embodiments. FIG. 6A—front cutaway view; FIG. 6B—front view; FIG. 6C—bottom view; FIG. 6D—perspective cutaway view; and FIG. 6E—perspective view.

FIGS. 7A-7E are various views of a signal generator, according to embodiments. FIG. 7A—front view; FIG. 7B—bottom view; FIG. 7C—side view; FIG. 7D—perspective cutaway view; and FIG. 7E—perspective view.

FIGS. 8A-8E are various views of a signal transducer, according to embodiments. FIG. 8A—front view; FIG. 8B—front cutaway view; FIG. 8C—front view with inner components shown in light lines; FIG. 8D—perspective cutaway view; and FIG. 8E—perspective view.

FIG. 9 is an illustration of an example system for airway treatment during use on an infant, according to embodiments.

FIG. 10 illustrates a system for calibration and noise reduction for airway treatment, according to embodiments.

FIG. 11 illustrates a method for calibration and noise reduction for airway treatment, according to embodiments.

FIGS. 12A-12C show experimental signals acquired during calibration and noise reduction in airway treatments, according to embodiments.

FIG. 13 illustrates a method 1300 of airway treatment, accordingly embodiments.

### DETAILED DESCRIPTION

Systems, devices and methods are described herein that are directed to pulmonary treatment. Embodiments describe herein provide for optimization of transfer of acoustic signals/waves to a user for treatment of pulmonary disorders.

As used in this specification, the singular forms “a,” “an” and “the” include plural referents unless the context clearly dictates otherwise. Thus, for example, the term “a network” is intended to mean a single network or a combination of networks.

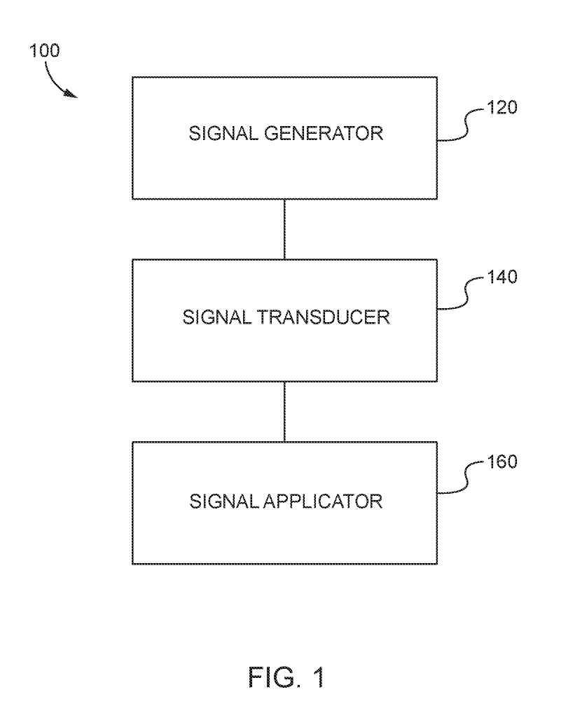

FIG. 1 is a schematic illustration of a system 100 for airway treatment, according to embodiments. The system 100 includes a signal generator 120 (sometimes also referred to as a “frequency generator”) to generate an electrical signal, a signal transducer 140 (sometimes also referred to as an acoustic transducer) to convert the electrical signal to an acoustic signal, and a signal applicator 160 (sometimes also referred to as an applicator, a head, or an application head) to apply acoustic signals to a patient. In some embodiments, the acoustic signals applied to the patient can be generated by the acoustic transducer 140. In some embodiments, the acoustic signals applied to the patient can be generated by the applicator 160 based on, for example, the acoustic signal generated by the acoustic transducer 140.

In some embodiments, the frequency generator 120 can include an analog frequency generator. In some embodiments, the frequency generator 120 can include a digital frequency generator. The frequency of the electrical signal generated by the frequency generator 120 can be adjustable so as to, for example, comply with different treatment protocols. In some embodiments, the output frequency of the frequency generator 120 can be set before each treatment. In some embodiments, the output frequency of the frequency generator 120 can be dynamically adjusted during treatment (e.g., using a feedback system to monitor the effect of treatment and adjust the output frequency based on the monitored effect).

The electrical signal generated by the frequency generator 120 can have various waveforms. In some embodiments, the frequency generator 120 can generate a sinusoidal wave. In some embodiments, the frequency generator 120 can generate a rectangular wave. The duty cycle of the rectangular wave can be from about 0.1 to about 0.9 (e.g., about 0.1, about 0.2, about 0.3, about 0.4, about 0.5, about 0.6, about 0.7, about 0.8, and about 0.9, including all values and sub ranges in between). In some embodiments, the frequency generator 120 can generate a square wave. In some embodiments, the frequency generator 120 can generate a sawtooth wave. In some embodiments, the frequency generator 120 can include an arbitrary waveform generator (AWG) to generate an electrical signal having an arbitrary waveform.

In some embodiments, the frequency generator 120 includes a function generator. In some embodiments, the frequency generator 120 includes a radio-frequency (RF) generator. In some embodiments, the frequency generator 120 includes a microwave generator. In some embodiments, the frequency generator 120 includes a pitch generator. In some embodiments, the frequency generator 120 includes an arbitrary waveform generator (AWG). In some embodiments, the frequency generator 120 includes a digital pattern generator.

In some embodiments, the frequency generator 120 can operate in continuous mode. In such embodiments, the electrical signal generated by the frequency generator 120 can include a continuous wave, and the acoustic wave converted from this electrical signal can be continuous. In some embodiments, the frequency generator 120 can operate in pulsed mode, generating pulses of electrical signals, and the acoustic wave converted from this electrical signal can include a train of acoustic pulses. The temporal pitch of the pulses train (i.e., time delay between adjacent acoustic pulses) can be, for example, from about 0.1 second to about 2 seconds (e.g., about 0.1 second, about 0.2 second, about 0.3 second, about 0.4 second, about 0.5 second, about 0.6 second, about 0.8 second, about 1.0 second, about 1.2 second, about 1.4 second, about 1.6 second, about 1.8 second, and about 2.0 seconds, including all values and sub ranges in between).

In some embodiments, the frequency generator 120 can include an amplifier (not shown in FIG. 1) to amplify the power of the electrical signal in any suitable manner. In some embodiments, the amplifier includes a power amplifier. In some embodiments, the amplifier includes a vacuum-tube amplifier. In some embodiments, the amplifier includes a transistor amplifier. In some embodiments, the amplifier includes a magnetic amplifier. In some embodiments, the amplifier includes an operational amplifier. In some embodiments, the amplifier includes a differential amplifier. In some embodiments, the amplifier includes a fully differential amplifier. In some embodiments, the amplifier includes a video amplifier. In some embodiments, the amplifier includes an oscilloscope vertical amplifier. In some embodiments, the amplifier includes a distributed amplifier. In some embodiments, the amplifier includes a switched mode amplifier. In some embodiments, the amplifier includes a negative resistance amplifier. In some embodiments, the amplifier includes a microwave amplifier. In some embodiments, the amplifier includes a metal-oxide-semiconductor field-effect transistor (MOSFET) amplifier. In some embodiments, the amplifier includes a switching amplifier. In some embodiments, the amplifier includes a combination of the above-mentioned amplifiers.

In some embodiments, the power of the electrical signal is amplified by a factor of about 2, of about 3, about 4, about 5, about 6, about 7 or above, including all values and sub ranges in between.

The acoustic transducer 140 can be based on various mechanisms. In some embodiments, the acoustic transducer 140 can include an electro-dynamic transducer, which can include a coil of wire suspended in a magnetic field. When an alternating electrical current is passed through the coil, mechanical forces can be developed between the coil's electromagnetic field and the field in which it is mounted (sometimes also referred to as an external field). The coil of wire is usually rigidly connected to a radiating diaphragm that is in turn resiliently mounted to an enclosure. This can hold the coil within the external field, but allows it to freely vibrate within this external field. The mechanical force developed between the coil's electromagnetic field and the external field can then cause the coil to move back and forth, vibrating the diaphragm and generating sound.

In some embodiments, the acoustic transducer 140 can include a magnetostrictive material for transduction. When a magnetostrictive material is placed in a magnetic field, its mechanical dimensions can change as a function of the strength of the magnetic field, which in turn can be used to generate sound.

In some embodiments, the acoustic transducer 140 can use a piezoelectric crystal, including but is not limiting to, Quartz, Rochelle Salt, and Ammonium Dihydrogen Phosphate (ADP), for transduction. Piezoelectric crystals can develop an electric charge between two surfaces of the crystals when the crystals are mechanically compressed, and they expand and contract in size in the presence of an applied electrical field. Therefore, by applying an external electrical field, the piezoelectric crystals can contract and expand, thereby causing, for example, a diaphragm to generate sound waves.

In some embodiments, the acoustic transducer 140 can include electrostrictive ceramics (sometimes also referred to as piezoelectric ceramics) for transduction. Electrostrictive materials, such as Barium Titinate and Lead Zirconate Titanate, can produce an electric charge when a mechanical stress is applied. Conversely, an electric field applied over piezoelectric ceramics can cause a change of physical dimensions of the piezoelectric ceramics, thereby generating acoustic waves.

In some embodiments, the acoustic transducer 140 is configured to receive the electrical signal from the signal generator 120, and is further configured to convert the electrical signal into an acoustic signal. In some embodiments, the signal transducer 140 can include an electromagnetic acoustic transducer (EMAT), which in turn includes a magnet and an electric coil. In some embodiments, the magnet can be a permanent magnet, which in other embodiments, the magnet can be an electromagnet.

In some embodiments, the signal transducer 140 can include an electric coil and a vibrating membrane, where application of the electrical signal to the coil generated a varying magnetic field that in turn causes the membrane to vibrate. In some embodiments, instead of a vibrating membrane, a magnetorestrictive material (e.g., cobalt) can be used that changes mechanical dimensions in the presence of the magnetic field, generating sound in the process. In some embodiments, piezoelectric crystals, such as Quartz, Rochelle Salt, or Ammonium Dihydrogen Phosphate (ADP) can be employed for electroacoustic transduction. In some embodiments, the signal transducer 140 can include electrostrictive ceramics for transduction.

In some embodiments, the acoustic signal has a frequency of about 20 Hz, of about 25 Hz, about 30 Hz, about 35 Hz, about 40 Hz, about 45 Hz, about 50 Hz, about 55 Hz, about 60 Hz, about 65 Hz, including all values and subranges in between.

In some embodiments, the acoustic transducer 140 generates an acoustic wave having a power from about 5 Watts to about 50 Watts (e.g., about 5 Watts, about 10 Watts, about 15 Watts, about 20 Watts, about 25 Watts, about 30 Watts, about 35 Watts, about 40 Watts, about 45 Watts, and about 50 Watts, including all values and sub ranges in between).

The connection or coupling between the acoustic transducer 140 and the applicator 160 can be achieved in various ways. In some embodiments, the acoustic transducer 140 is operably coupled to the signal applicator 160 via an acoustic waveguide that can guide acoustic waves. In some embodiments, the acoustic waveguide includes a tube. In some embodiments, the acoustic waveguide includes a pipe. In some embodiments, the acoustic waveguide includes an acoustic transmission line. In some embodiments, the acoustic waveguide includes any other acoustic transmission devices.

In some embodiments, the acoustic waveguide is sufficiently flexible, such that during use, a user has some range of motion that would be otherwise inhibited by use of rigid transmission line. In some embodiments, the acoustic waveguide can be reinforced to provide some resistance to bending, breaking, and/or kinking. For example, a spiral metal coil can be formed on at least a portion of the acoustic waveguide for reinforcement. In some embodiments, the acoustic waveguide can be relatively thin and reinforced by an exterior coil.

The acoustic waveguide coupling the acoustic transducer 140 and the applicator 160 can be of any suitable length to permit convenient use by a practitioner/user. In some embodiments, the length of the acoustic waveguide can be about 1 foot to about 10 feet (e.g., about 1 foot, about 2 feet, about 3 feet, about 4 feet, about 5 feet, about 6 feet, about 7 feet, about 8 feet, about 9 feet, about 10 feet, including any values and sub ranges in between).

In some embodiments, the length of the acoustic waveguide is selected based on the wavelength (or frequency) of the acoustic signal. In some embodiments, the length of the acoustic waveguide is selected based on a need to account for (destructive) reflecting signals derived from the acoustic signal.

In some embodiments, the signal transducer 140 includes an adapter (not shown in FIG. 1) for interfacing the acoustic waveguide (discussed in more details below). For example, the signal transducer 140 can include a vibration chamber holding a vibrating membrane that can generate acoustic waves. The adapter can be configured to change the diameter (or cross sectional area) of the chamber to substantially match the diameter (or cross-section area) of acoustic waveguide.

In some embodiments, the adapter of the signal transducer 140 includes a flute-like mechanism that reduces the diameter of the vibration chamber to match the diameter of the acoustic waveguide. In some embodiments, the reduction in diameter can be about 2-fold to about 10-fold (e.g., about 2-fold, about 3-fold, about 4-fold, about 5-fold, about 6 fold, about 7-fold, about 8-fold, about 9-fold, about 10-fold, including any values and sub ranges in between).

In some embodiments, the adapter includes a logarithmic reduction flute that reduces the diameter of the vibration chamber from about 4 inches to about 1 inch to permit a 1-inch diameter tubing to be attached to the signal transducer 140. In some embodiments, the adapter includes a linear reduction flute that reduces the diameter of the vibration chamber to match the diameter of the acoustic waveguide. In some embodiments, the adapter includes a polynomial reduction flute that reduces the diameter of the vibration chamber to match the diameter of the acoustic waveguide. For example, the diameter of the adapter D can be determined by D=a(d)^{-b}, where d is the distance from the starting point of the adapter, a and b are constants that can affect the speed of diameter reduction of the adapter.

In some embodiments, the acoustic waveguide is further configured to not only transmit the acoustic signal, but also air pressure from the signal transducer 140 to the signal applicator 160. In general, by transmission of the acoustic signal, the acoustic waveguide can transmit a change (or variation) of pressure from one end to the other. In this manner, when air inside the acoustic waveguide is set into motion at one end (e.g., by a vibrating membrane, or other mechanism), the air in the acoustic waveguide moves back and forth at the same frequency at which the membrane/other mechanism is vibrating. This can result in the movement of an adapter at the other end, thereby generating another acoustic signal (described below). Further, since the acoustic waveguide can be flexible, the acoustic waveguide itself can vibrate due to the change in air pressure acting as “percussion” on the adapter at the other end. Since the adapter at the other end usually does not convert 100% of the air pressure change into sound, the rest can be absorbed by the body of the adapter itself and makes the adapter move (e.g., vibrate) in the air. The combination of these effects, when accounted for, can result in optimal transmission.

The acoustic waveguide can have various cross sectional shapes. In some embodiments, the acoustic waveguide can have a round cross section. In some embodiments, the acoustic waveguide can have a rectangular (including square) cross section. In some embodiments, the acoustic waveguide can have an elliptical cross section. In some embodiments, the acoustic waveguide can have any other cross sectional shape.

In some embodiments, the adapter of the signal transducer 140 is a first adapter, and the signal applicator 160 includes a second adapter for receiving the acoustic waveguide. In some embodiments, the second adapter is configured to match the diameter (or cross-section area) of the acoustic waveguide with the diameter (or cross-section area) of a vibrating membrane of the signal applicator 160 (explained below).

In some embodiments, the second adapter in the applicator 160 includes a funnel-like or flute-like mechanism that increases the diameter of the vibration chamber to match the diameter of the vibrating membrane of the acoustic transducer 140. In some embodiments, the increase in diameter is about 2-fold to about 10-fold (e.g., about 2-fold, about 3-fold, about 4-fold, about 5-fold, about 6-fold, about 7-fold, about 8-fold, about 9-fold, or about 10-fold, including any values and sub ranges in between).

In some embodiments, the adapter includes a logarithmic flute that increases the diameter of the vibration chamber from about 1 inch to about 2 inches. A logarithmic flute can generally be any funnel that is used to reduce (or increase) the size of a tube in a shape that uses a mathematical logarithmic or polynomial function to calculate the change in diameter of the funnel. In some embodiments, the logarithmic flute resembles the end of a trumpet. In some embodiments, the logarithmic flute resembles the end of a cone.

In some embodiments, the size/other specifications of the vibrating membrane and/or the flute-like mechanisms can be selected based on the target patient population. In some embodiments, the diameter increase affected by the adapter is based on the vibrating membrane of the signal applicator 160. In some embodiments, the recessed nature of the vibrating membrane of the signal applicator 160 provides the space required for air exchange, and to allow for vibration of the vibrating membrane of the signal applicator without contacting the skin of the patient.

In some embodiments, the first adapter, the acoustic waveguide, and the second adapter form a substantially airtight connection (also referred to as an airtight chamber, or sealed chamber) between the signal transducer 140 and the signal applicator 160. In this manner, attenuation or loss of acoustic signal can be minimized.

In some embodiments, the airtight connection is filled with any suitable fluid that can transmit and/or otherwise permits transmission of acoustic waves. As used herein, the word “fluid” is used to indicate air, liquid, gas, foam, fluid gels, combinations thereof, and/or any other material/medium used within the airtight connection. For example, in some embodiments, the airtight connection is filled with air. In some embodiments, the airtight connection is filled with nitrogen. In some embodiments, the airtight connection is filled with carbon dioxide. In some embodiments, the airtight connection is filled with any other gas or gas mixture that can transmit acoustic waves.

In some embodiments, the airtight connection is filled with liquid that can transmit acoustic waves. In some embodiments, the filled liquid includes water. In some embodiments, the filled liquid includes oil.

In some embodiments, the first adapter is removable and/or replaceable from the signal transducer 140, such that adapters of different sizes can be employed. In some embodiments, the second adapter is removable and/or replaceable from the signal applicator 160. In this manner, a host of conduit sizes as well as sizes of the vibrating membrane(s) can be accommodated.

In some embodiments, the signal applicator 160 is configured to receive the acoustic signal from the signal transducer 140, and is further configured to apply the acoustic signal to a patient during use. In some embodiments, the signal applicator 160 can be configured to apply the acoustic signal to the airways of the user, such as by placement on or near the chest of the patient.

In some embodiments, the signal applicator 160 is constructed of a lightweight material. In some embodiments, the signal applicator 160 is made of any suitable plastic materials. In some embodiments, the signal applicator 160 is made of polycarbonate. In some embodiments, the signal applicator 160 is constructed of any suitable material, such that the signal applicator 160 can sustain a drop test at a height of about 1 meter, of about 2 meters, of about 3 meters, of about 4 meters, including all values and sub ranges in between. In this manner, heavier components can be located in the signal generator 120 and the signal transducer 140, thereby permitting safe use of the signal applicator 160 with infants and other young patients that may not bear significant weight on their chest.

In some embodiments, the signal applicator 160 is configured to receive the acoustic signal from the signal transducer 140 as a first acoustic signal, and is further configured to generate a second acoustic signal for application to the user. The second acoustic signal is based on the first acoustic signal. In some embodiments, the signal applicator 160 includes a vibrating membrane that, upon stimulation by the first acoustic signal, generates the second acoustic signal. In some embodiments, where the signal transducer 140 includes a vibrating membrane configured to generate the first acoustic signal as described above, the vibrating membrane of the signal applicator can have the same resonant frequency as the vibrating membrane of the signal transducer. In some embodiments, the second acoustic signal has different amplitude from the amplitude of the first acoustic signal. In some embodiments, the amplitude of the second acoustic signal is lower than the amplitude of the first acoustic signal by a predetermined amount. In some embodiments, the amplitude of the second acoustic signal is greater or lower than the amplitude of the first acoustic by about 5% to about 30% (e.g., about 5%, about 10%, about 10%, about 10%, about 10%, or about 30%, including any values and sub ranges in between).

In some embodiments, the frequency of the second acoustic signal is different from the first acoustic signal. In some embodiments, the frequency of the second acoustic signal is higher than the frequency of the first acoustic signal. In some embodiments, the frequency of the second acoustic signal is lower than the frequency of the first acoustic signal. In some embodiments, the different between the frequency of the first acoustic signal and the frequency of the second acoustic signal is about 5% to about 30% of the frequency of the first acoustic signal (e.g., about 5%, about 10%, about 10%, about 10%, about 10%, or about 30%, including any values and sub ranges in between). In some embodiments, the different between the frequency of the first acoustic signal and the frequency of the second acoustic signal is about 5 Hz to about 500 Hz (e.g. about 5 Hz, about 20 Hz, about 50 Hz, about 100 Hz, about 200 Hz, or about 500 Hz, including any values and sub ranges in between). In these cases, the addition of a lightweight membrane to the signal applicator 160 permits favorable application for infants and other young patients.

In some embodiments, the vibrating membrane of the signal applicator 160 is recessed from an interface/head of the signal applicator 160 that is applied to the patient. In this manner, the membrane is not in direct contact with the patient and is free to vibrate during use. In some embodiments, the signal application 160 includes a sealed tube, which has an end cap as the interface. The application membrane in this case is recessed from the end cap. In some embodiments, the interface of the signal applicator 160 has an open-air configuration. In this case, the signal applicator 160 can include a tube holding the applicator membrane but without an end cap. The applicator membrane can be still kept away from the patient or user during use by the wall of the tube.

In some embodiments, the interface/head of the signal applicator 160 is replaceable, such that a new head can be employed for each new patient, thereby preventing contamination issues. In some embodiments, the vibrating membrane of the signal applicator 160 is recessed within the head, and is replaced when the head is replaced. In this manner, replacing the vibrating membrane minimizes the possibility of contamination in the conduit/tubing due to buildup on the vibrating membrane, thereby reducing possibility of infection in subsequent users.

In some embodiments, the system 100 can further include at least a processor and a memory (not shown). In some embodiments, the system 100 can also include a database, although it will be understood that, in some embodiments, the database and the memory can be a common data store. In some embodiments, the database can constitute one or more databases. Further, in other embodiments (not shown), at least one database can be external to the system 100 and can be accessed via wired or wireless connections (e.g., Internet or any other means). In some embodiments, the system 100 can also include an input/output (I/O) component 168, which can depict one or more input/output interfaces, implemented in software and/or hardware, for other entities to interact directly or indirectly with the system 100, such as a human user of the system 100.

The memory and/or the database can independently be, for example, a random access memory (RAM), a memory buffer, a hard drive, a database, an erasable programmable read-only memory (EPROM), an electrically erasable read-only memory (EEPROM), a read-only memory (ROM), Flash memory, and/or so forth. The memory and/or the database can store instructions to cause the processor to execute modules, processes and/or functions associated with the system 100 such as, for example, modules for interfacing with and/or controlling operation of the signal generator 120, the signal transducer 140, and/or the signal applicator 160. Generally, each module can independently be a hardware module and/or a software module (implemented in hardware, such as in the processor). In some embodiments, at least some of the modules can be operatively coupled to each other.

The processor can be, for example, a general purpose processor, a Field Programmable Gate Array (FPGA), an Application Specific Integrated Circuit (ASIC), a Digital Signal Processor (DSP), and/or the like. The processor can be configured to run and/or execute application processes and/or other modules, processes and/or functions associated with the system 100 and/or a network associated therewith.

In some embodiments, the system 100 can also include a communication module that can be configured to facilitate network connectivity for the system. For example, the communication module can include and/or enable a network interface controller (NIC), wireless connection, a wired port, and/or the like. As such, the communication module can establish and/or maintain a communication session with other devices/systems.

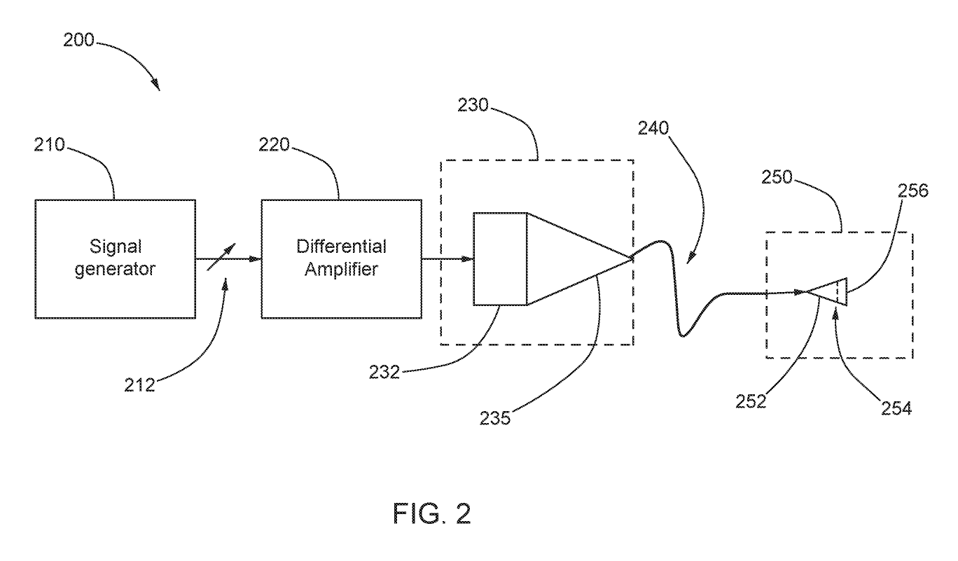

FIG. 2 illustrates an example system 200, according to embodiments. The system 200 can be structurally and/or functionally similar to the system 100. Components of the system 200, unless indicated otherwise, can be structurally and/or functionally similar to similarly named components of the system 100. The system 200 includes a frequency generator 210, a calibration component 212, and a differential amplifier 220, which in combination can be similar to the signal generator 120. The generator 210 is configured for generating an electrical signal, such as, for example, a sine wave from 20 to 65 Hz with amplitude from 0 to 100%. The calibration component 212 is configured for calibrating the system 200 (see details below). The differential amplifier 220 is configured for amplifying the generated electrical signal.

The system 200 also includes a transducer 230 configured for transforming the electrical signal into an acoustic signal, which can be similar to the signal transducer 140. The transducer 230 includes an electroacoustic converter 232, which can include a vibration chamber with a vibrating membrane. The transducer 230 also includes a flute/adapter 235, which can be a logarithmic flute configured to match the diameter of the vibration chamber at one end (e.g., about 4 inches), and to match the diameter of an acoustic waveguide 240 connected to the flute 235.

The acoustic waveguide 240, in some embodiment, can be selected to optimize the transmission of the acoustic signal, and to minimize attenuation. The acoustic waveguide 240 can be thin and light, and can further include a relatively stronger spiral/coiled material (e.g., any suitable plastic, metal, and combinations thereof) on an outer layer of the acoustic waveguide 240 to impart resistance to excessive bending/kinking. Maintaining the spiral/coiled material on the outer layer permits an inner layer of the acoustic waveguide to be smooth and obstruction free for passage of the acoustic signal.

In some embodiments, the acoustic waveguide 240 includes a tube having a diameter from about 12 mm to about 40 mm (e.g., about 12 mm, about 15 mm, about 18 mm, about 20 mm, about 25 mm, about 30 mm, about 35 mm, or about 40 mm—including any values and sub ranges in between). In some embodiments, the tube can have a diameter of about 25 mm or less (e.g., about 25 mm, about 25 mm, about 25 mm, or less, including any values and sub ranges in between).

The system 200 also includes an acoustic applicator 250 configured for receiving the acoustic signal from the acoustic waveguide 240, and further configured for applying the acoustic signal, or a variant thereof, to the user and/or the patient.

In some embodiments, the acoustic applicator 250 can be similar to the signal applicator 160. The connections of the acoustic waveguide 240 with the transducer 230 and the acoustic applicator 250 can be airtight to prevent acoustic signal loss. The airtight connection can also be filled with air, liquid(s), or any other material that can facilitate transmission of acoustic signals.

The acoustic applicator 250 includes an adapter/acoustic amplification chamber 252, a vibrating membrane 254, and an applicator interface 256. The amplification chamber 252 can amplify the acoustic signal received from the acoustic transducer 230 via the acoustic waveguide 240. The vibration membrane 254 can generate a second acoustic signal to be applied to the patient based on the acoustic signal amplified by the amplification chamber 252. The applicator interface 256 can be in direct contact with the patient during use to transmit the second acoustic signal to the patient.

In some embodiments, the acoustic amplification chamber 252 is a logarithmic flute configured to increase the internal diameter such as, for example, from about 1 inch to about 2 inches. This allows for relatively more space for the air exchange that is needed to create the vibration of the membrane 254. The membrane 254, in some embodiments, can be a thin membrane made of silicone that vibrates to the frequency output by the transducer 230. In some embodiments, the membrane 254 can include a center in rigid plastic and an outer part made of neoprene type of material. In some embodiments, the membrane 254 can include any other flexible material to make a speaker diaphragm, such as paper, plastic, or metal. In some embodiments, the membrane 254 can be recessed in the acoustic amplification chamber 252, so that it can vibrate freely without touching the user.

In some embodiments, the membrane 254 can be removable and/or replaceable for different users or for implementing different protocols for the same user. In some embodiments, the application 250 can be removable and/or replaceable from the acoustic waveguide 240 for different users or for implementing different protocols for the same user.

In some embodiments, the assembly of the acoustic waveguide 240 and the applicator 250 can be removable and/or replaceable from the acoustic transducer 230. In other words, the connection between the acoustic transducer 230 and the acoustic waveguide 240 can be removed and reinstalled freely.

In some embodiments, both the acoustic waveguide 240 and the applicator 250 can be removable and/or replaceable. For example, the acoustic waveguide 240 can be removable and/or replaceable from both the acoustic transducer 230 and the applicator 250. In this manner, a user can replace the acoustic waveguide 240 but keep the applicator 250. Alternatively, the user can replace the applicator 250 but keep the acoustic waveguide 240. In yet another option, the user can replace both the applicator 250 and the acoustic waveguide 240.

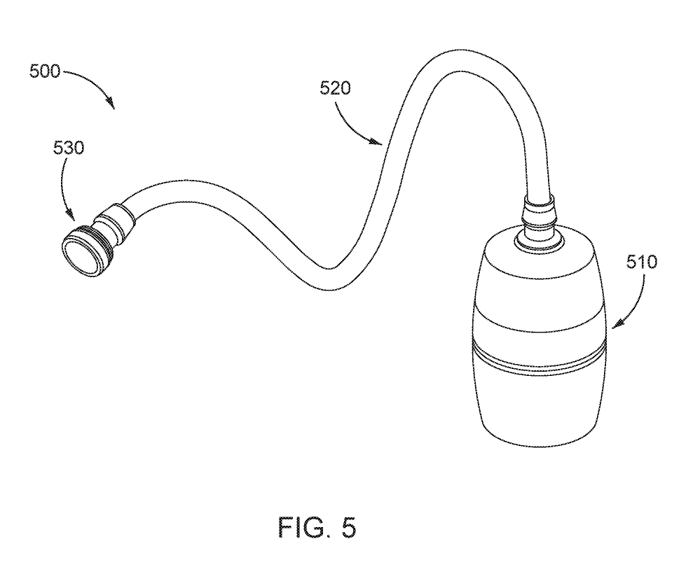

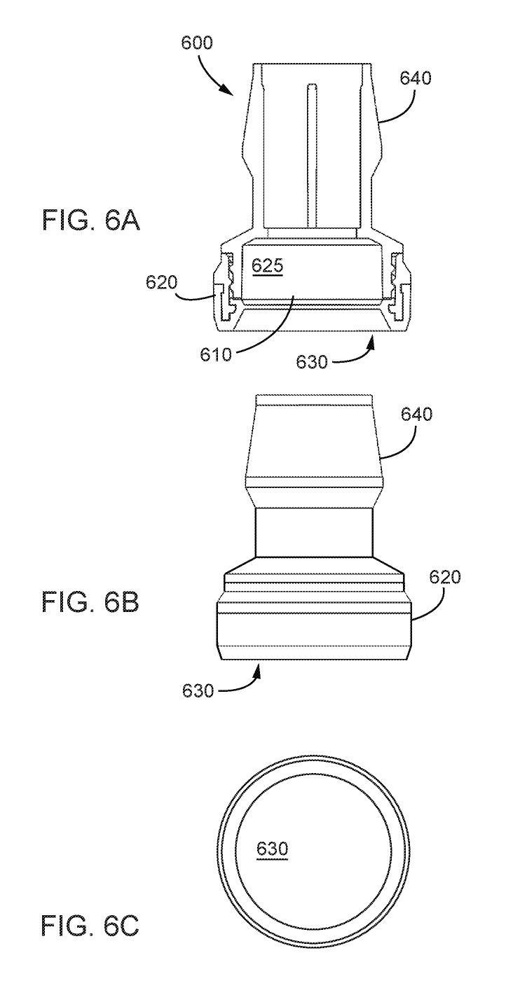

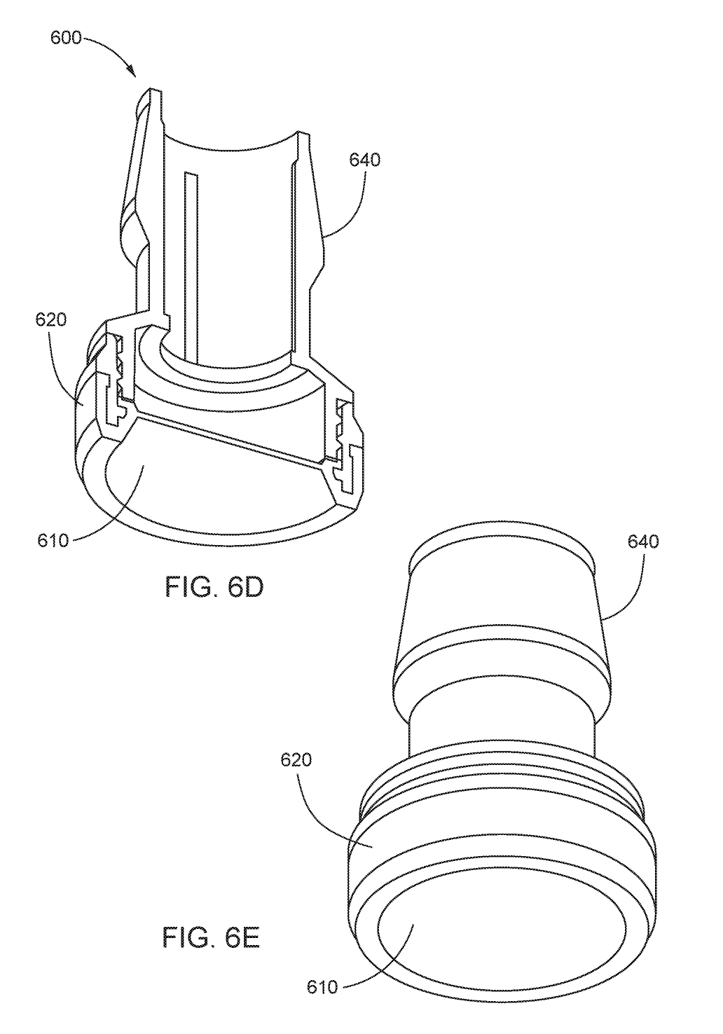

In some embodiments, the applicator interface 256 can simply be an air gap (e.g., see FIGS. 6A-6E). In some embodiments, the applicator interface 256 can include a cap-like or a plate-like element fixed to the amplification chamber 252. In some embodiment, the applicator interface 256 can include soft materials to increase comfort level for the patient during treatment. In some embodiments, the exterior shape of the applicator interface 256 can substantially match the general contour of the chest of the patient. In some embodiments, the exterior surface of the applicator interface 256 can include a memory material such as memory foam, a shape-memory polymer, and/or the like, to improve and/or enhance the surface area of the matching between the applicator interface 256 and the chest of the patient during treatment.

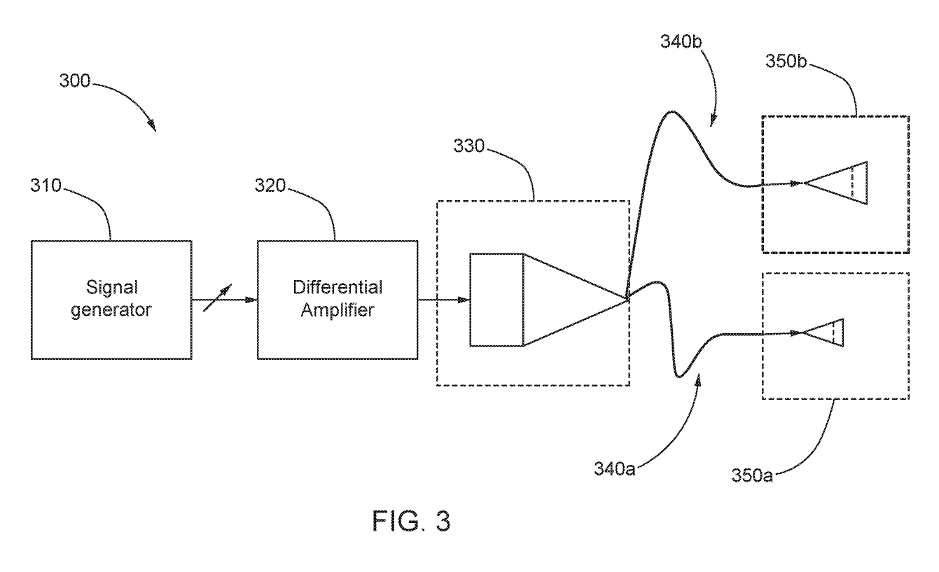

FIG. 3 shows a schematic of a system 300 including two applicators. The system 300 includes a frequency generator 310 to generate an electrical signal, an amplifier 320 to amplify the electrical signal, and an acoustic transducer 330 to convert the amplified electrical signal into an acoustic signal (i.e. acoustic wave). Two flexible acoustic waveguides 340a and 340b are coupled to the acoustic transducer 330 to transmit the acoustic signal to applicators 350a and 350b, respectively.

In some embodiments, the two acoustic waveguides 340a and 340b can have different parameters to implement different protocols. Similarly, the two applicators 350a and 350b can also have different parameters to implement different protocols. For example, the two applicators 350a and 350b can have different diameters. In another example, the two applicators 350a and 350b can have different materials for the vibrating membranes. In this case, the two membranes can have different resonant frequency and accordingly can generate acoustic waves of different frequencies.

In some embodiments, the two applicators 350a and 350b can be substantially the same. In this case, the dual applicator configuration can allow treatment of multiple patients simultaneously. In some embodiments, the system 300 can include more than two applicators (e.g., three applicators, four applicators, five applicators, or even more).

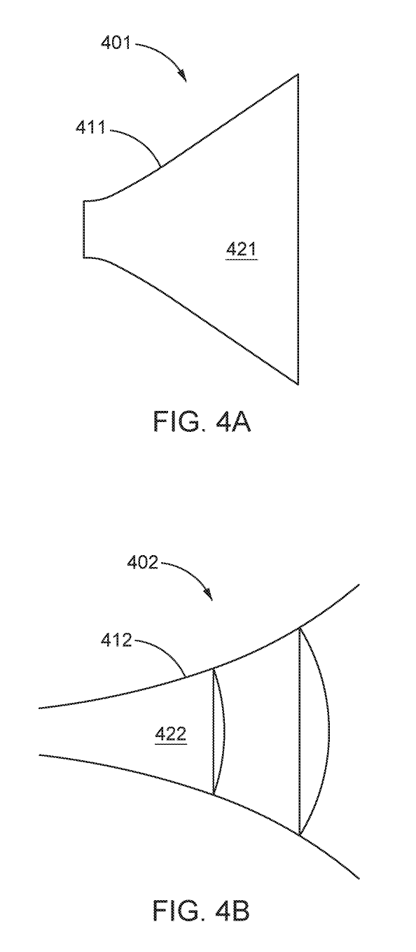

FIGS. 4A and 4B are illustrations of adapters 401 and 402 that can be used in airway treatment systems (e.g., as adapters 235 and/or 252 as shown in FIG. 2 and described above), according to embodiments. The adapter 401 includes a side wall 411 defining a chamber 421, in which acoustic waves can be amplified or attenuated. The side wall 411 has a linear shape (i.e. a straight line). The adapter 402 includes a side wall 412 defining a chamber 422, in which acoustic waves can be amplified or attenuated. The side wall 412, in contrast, has a nonlinear shape. In some embodiments, at least a portion of the side wall 412 can be parabolic. In some embodiments, at least a portion of the side wall 412 can be hyperbolic. In some embodiments, at least a portion of the side wall 412 can be elliptical. In some embodiments, at least a portion of the side wall 412 can be polynomial. In some embodiments, at least a portion of the side wall 412 can be exponential.

In some embodiments, the aperture shape (also referred to as the opening shape) of the adapters 401 and/or 402 can be round. In some embodiments, the aperture shape (also referred to as the opening shape) of the adapters 401 and/or 402 can be rectangular. In some embodiments, the aperture shape (also referred to as the opening shape) of the adapters 401 and/or 402 can be square. In some embodiments, the aperture shape (also referred to as the opening shape) of the adapters 401 and/or 402 can be elliptical.

In some embodiments, the angle at which the waveguide connects to the adapters 401 and/or 402 can be about 1 degree to about 60 degrees (e.g. about 1 degree, about 5 degrees, about 10 degrees, about 15 degrees, about 20 degrees, about 25 degrees, about 30 degrees, about 35 degrees, about 40 degrees, about 45 degrees, about 50 degrees, about 55 degrees, or about 60 degrees, including any values and sub ranges in between).

FIG. 5 shows a schematic of an apparatus 500 for airway treatment. The apparatus 500 includes an acoustic transducer 510, a flexible tube 520, and an applicator 530. The flexible tube 520 can include connectors at one or both ends so as to facilitate convenient removal or replacement of the applicator 530 and/or the tube 520. In some embodiments, the connectors include barbed connectors. In some embodiments, the connectors include threaded connectors. In some embodiments, the connectors include swivel connectors. In some embodiments, the connectors include any other connectors. Accordingly, the signal generator 510 and the signal applicator 530 can include corresponding mating connectors.

The signal transducer 510, in the illustrated embodiment, is barrel-shaped, and can include a vibrating membrane. In practice, other shapes can also be used for the signal transducer. In one example, the signal transducer 510 can be configured into a box. In another example, the signal transducer 510 can be configured into a cylinder. In yet another example, the signal transducer 510 can be configured into a sphere or a portion of a sphere.

FIGS. 6A-6E are various views of an acoustic signal applicator 600 that can be used in systems described herein, according to embodiments. FIG. 6A shows a front cutaway view of the applicator 600. FIG. 6B shows a front view of the applicator 600. FIG. 6C shows a bottom view of the applicator 600. FIG. 6D shows a perspective cutaway view of the applicator 600. FIG. 6E shows a perspective view of the applicator 600.

The applicator 600 includes an applicator chamber 625 defined by a chamber wall 620, an applicator membrane 610, a handle portion 640, and an applicator interface 630. The applicator chamber 625 receives acoustic signals from, for example, a transducer. In some embodiments, the applicator chamber 625 can amplify the received acoustic signals. In some embodiments, the applicator chamber 625 can attenuate the received acoustic signals. The applicator membrane 610 can be excited by acoustic signals to generate another set of acoustic signals. The applicator interface 630 applies the acoustic signal generated by the applicator membrane 610 to a patient. The handler portion 640 can facilitate handling of the applicator 600. For example, a practitioner can hold the handler portion 640 during treatment.

In some embodiments, the applicator membrane 610 can substantially seal the applicator chamber 620 so as to form an airtight connection with other components in a treatment system (e.g., transducer).

In some embodiments, the applicator membrane 610 is recessed from the applicator interface 630 (see, e.g., FIG. 6A and FIG. 6D). In this case, the applicator membrane 610 can vibrate freely without touching the patient during treatment. In some embodiments, the distance between the applicator membrane 610 and the applicator interface 630 can be about 0.5 inch of diameter to about 5 inches (e.g., about 0.5 inch, about 1 inch, about 1.5 inch, about 2 inches, about 2.5 inches, about 3 inches, about 3.5 inches, about 4 inch, about 4.5 inches, and about 5 inches, including all values and sub ranges in between).

In some embodiments, the chamber wall 620 can be removable from the handler portion 640 by, for example, unscrewing the chamber wall 620 from the handler portion 640. In some embodiments, a new membrane can be installed by using a new chamber wall 620 including with the new membrane fixed on the chamber wall 620.

Although illustrated here as a threaded connection between the chamber wall 620 and the rest of the signal applicator 600, any other suitable connecting means can be employed such as, for example, luer locks, tight-fit, and/or the like.

The chamber 625 can have any suitable diameter for application to a user, such as, for example, about 1 inch to about 10 inches (about 1 inch, about 2 inches, about 3 inches, about 4 inches, about 5 inches, about 6 inches, about 7 inches, about 8 inches, about 9 inches, or about 10 inches, including any values and sub ranges in between). The diameter can be increased/decreased by replacement of the chamber wall 620 with another of the desired diameter.

In some embodiments, the cross section of the chamber 625 can be round. In some embodiments, the cross section of the chamber 625 can be rectangular. In some embodiments, the cross section of the chamber 625 can be square. In some embodiments, the cross section of the chamber 625 can be elliptical.

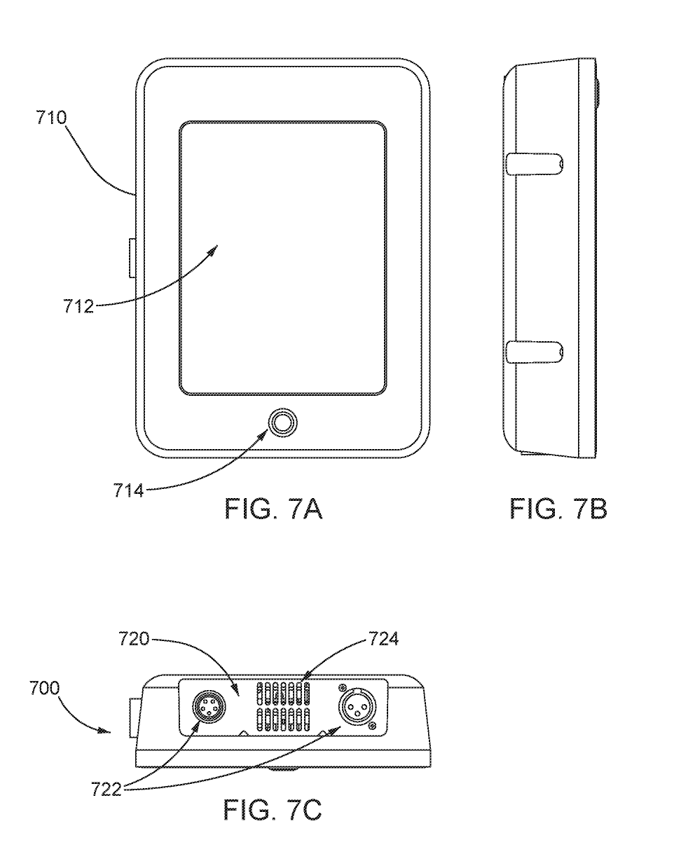

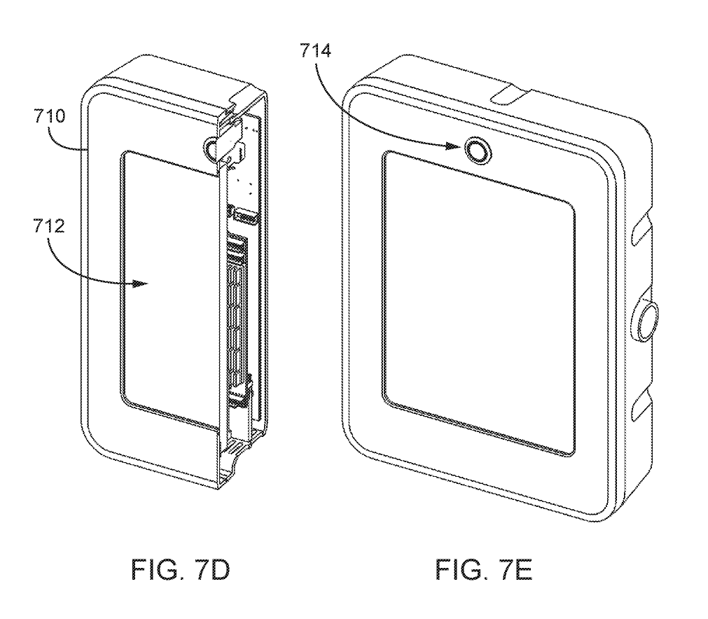

FIGS. 7A-7E are various views of a signal generator 700 that can be used in systems described herein, according to embodiments. More specifically, FIG. 7A shows a front view of the signal generator 700. FIG. 7B shows a bottom view of the signal generator 700. FIG. 7C shows a side view of the signal generator 700. FIG. 7D shows a perspective cutaway view of the signal generator 700. FIG. 7E shows a perspective view of the signal generator 700.

The signal generator 700 includes a controller 710 that further includes a controller interface 712 and a button 714 allowing interactive communication between a user and the treatment system including the signal generator 700. The signal generator 700 also includes casing 720, which houses ports and connectors 722 for power and/or information exchange and vents 724 for air flow and cooling.

In some embodiments, the controller interface 712 includes a touch screen so the user can conveniently control the signal generator (and/or other components in treatment systems including the signal generator 700). In some embodiments, the controller interface 712 can include other types of interface such as a key board. In some embodiments, the controller interface 712 can include a voice command device so as to receive voice commands from the user. In some embodiments, the controller interface 712 can include gesture recognition devices so as to recognize gestures from users and generate control signals based on the recognized gestures of the users.

In some embodiments, the button 714 can be used for turning on and/or off of the signal generator 700. In some embodiments, the button 714 can be used for turning on and/or off of the controller interface 712 and the signal generator 700 may have another power button.

In some embodiments, the controller 710 can be configured to be disposed on the applicator (e.g., the applicator illustrated in FIGS. 6A-6E). In some embodiments, the controller 710 can be configured to be disposed on the side wall of the applicator. In some embodiments, the controller 710 can be configured to be disposed on the end of the applicator. These configurations allow the user to conveniently reach the controller 710 that is close to his or her hand, thereby improving control of the system.

In some embodiments, the controller interface 712 can be configured to control one or more of the treatment parameters. In some embodiments, the controller interface 712 can control the initiation of the treatment. In some embodiment, the controller interface 712 can cause a pause of the treatment for a predetermined amount of time or until a resume command is received. In some embodiment, the controller interface 712 can stop the treatment.

In some embodiments, the controller interface 712 can control the frequency of the acoustic signal. In some embodiments, the controller interface 712 can control the power of the acoustic signal. In some embodiments, the controller interface 712 can control the amplitude of the acoustic signal. In some embodiments, the controller interface 712 can control the repetition rate of the acoustic signal.

In some embodiments, the controller interface 712 can automatically implement the interval treatment protocols. In some embodiments, the controller interface 712 can implement variable frequency and amplitude treatment protocols. In some embodiments, the controller interface 712 can control wired/wireless connectivity to receive/transmit patient medical file(s) to/from another device, and/or the like.

In some embodiments, the controller 710 can be controlled by another device. In some embodiments, the controller 710 can be controlled by a smartphone. In some embodiments, the controller 710 can be controlled by a remote controller.

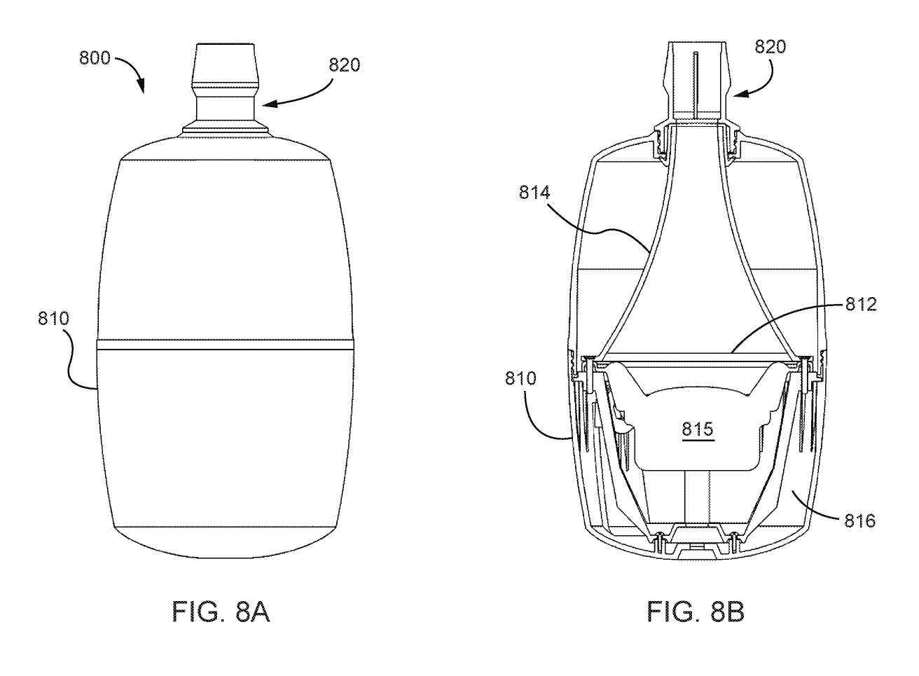

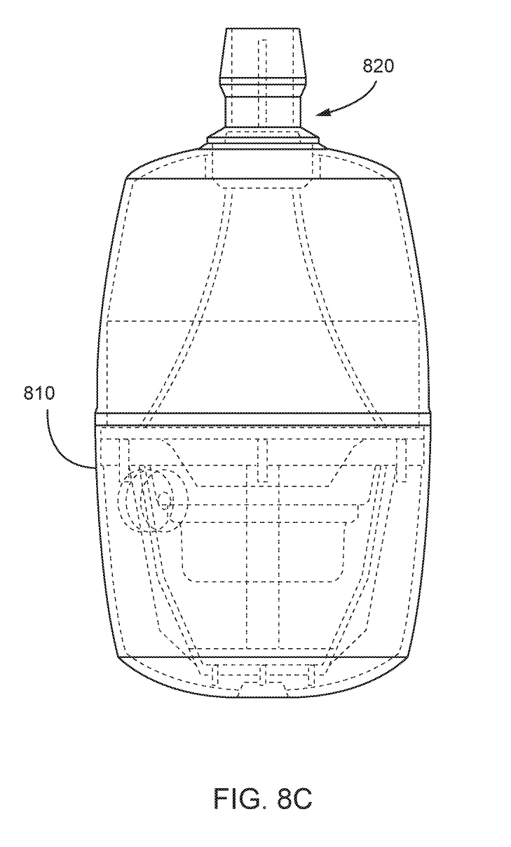

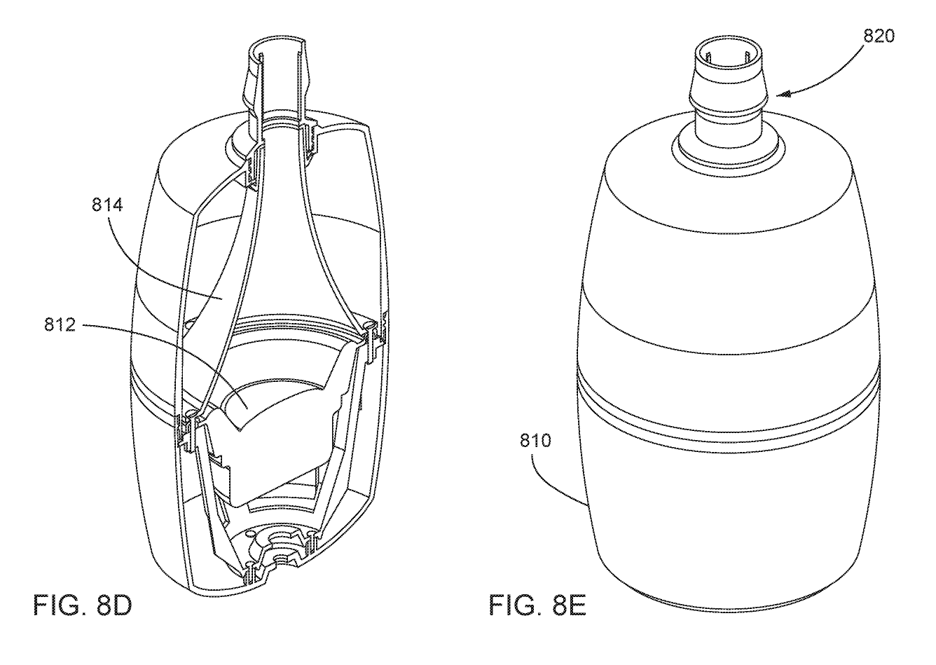

FIGS. 8A-8E are various views of a signal transducer 800 that can be used in systems described herein, according to embodiments. More specifically, FIG. 8A shows a front view of the transducer 800. FIG. 8B shows a front cutaway view of the transducer 800. FIG. 8C shows a front view of the transducer 800 with inner components shown in light lines. FIG. 8D shows a perspective cutaway view of the transducer 800. FIG. 8E shows a perspective view of the transducer 800.

As best illustrated in FIGS. 8B and 8D, the signal transducer 800 includes a transducer chamber 810 with a transducer membrane 812 suspended by the transducer chamber 810 to generate acoustic signals. In some embodiments, the thickness of the transducer membrane 812 is about 200 μm to about 2 mm (e.g., about 200 μm, about 400 μm, about 600 μm, about 800 μm, about 1.0 mm, about 1.2 mm, about 1.4 mm, about 1.6 mm, about 1.8 mm, or about 2 mm, including any values and sub ranges in between). The transducer membrane 812 is connected to a speaker 815, which is disposed above an array of brackets 816 to hold the speaker 815 in place. The lower part where the 815 is contained can also act as an air cushion for the kickback of the speaker membrane 812. In some embodiments, the transducer chamber 810 is a sealed air tight space.

The signal transducer 800 also includes an adapter 814 in the transducer chamber 810 to modulate the acoustic signals generated by the transducer membrane 812. The signal transducer 800 also includes a connector 820 that can couple the signal transducer 800 to other components in a treatment system, such as an acoustic waveguide.

In some embodiments, the adapter 814 includes a logarithmic flute adapter as illustrated in FIG. 8B. In some embodiments, the adapter 814 includes a linear flute adapter. In some embodiments, the adapter 814 includes any other adapter.

The lower portion of the transducer chamber 810 including the transducer membrane 812 can be releasably coupled to other portion of the transducer chamber 810. This coupling can be achieved by, for example, a threaded connection. In this manner, the transducer membrane 812 can be replaced, and the inner space of the signal transducer 800 can be periodically cleaned.

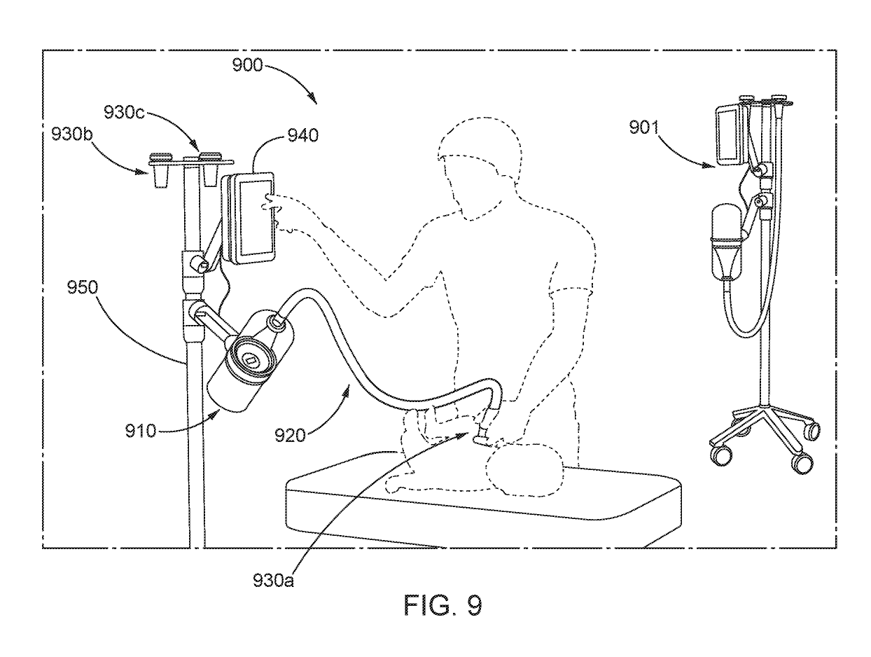

FIG. 9 illustrates a system 900 for airway treatment, according to embodiments. The system 900 includes a transducer 910 to generate acoustic signals, a tube 920 to transmit the acoustic signals to an applicator 930a, which can either apply the acoustic signals to a patient or generate another acoustic signal for treatment. The system 900 also includes a controller 940, which has a touch screen for the practitioner to conveniently control the treatment. In some embodiments, the controller 940 may include a frequency generator to generate the electrical signal for the transducer 910 to convert into acoustic signals. In some embodiments, the frequency generators can be coupled directly to the transducer 910.

The controller 940 and the transducer 910 are supported by a stand 950, which can have a swivel mechanism to permit convenient manipulation of the transducer 950 and/or the applicator 930. By placing the larger and usually bulkier components on the stand 950, the footprint and weight of the signal applicator 930 can be minimized, thereby providing for safe and convenient use on infants or aged group of people.

The stand 950 also includes a holder to hold additional applicators 930b and 930c. During treatment or to accommodate different treatments, these applicators 930b and 903c can be removed from the stand 910 and replace the applicator 930a.

In some embodiments, the controller 940 includes the signal generator and the electrical signal is transmitted to the transducer 910 via wires at least partially disposed within the stand 910. In some embodiments, the controller 940 includes the signal generator and the electrical signal is transmitted to the transducer 910 via wireless transmission. Examples of wireless transmission methods include, but not limited to, WiFi, 3G, 4G, Bluetooth, radio frequency (RF), or any other methods.

A separate airway treatment device 901 is illustrated in FIG. 9. The treatment device 901 includes a wheeled stand to hold other components, such as the transducer, the tube, the controller, and the applicator. In this manner, the device 901 can be highly transportable so as to allow convenient use at various locations.

Embodiments disclosed herein are also directed to minimizing and/or substantially eliminating performance variation in the systems and devices for pulmonary treatment described above. In some embodiments, a calibration apparatus/method is provided. In some embodiments, the calibration apparatus/method encompasses adjustment of the output of the differential amplifier (e.g., 220 shown in FIG. 2), and further encompasses standardizing all systems and devices for pulmonary treatment to substantially the same final vibration and sound level. In some embodiments, the calibration apparatus/method disclosed herein includes addition of dynamic fan control to reduce background noise created by the cooling of the systems and devices for pulmonary treatment. In some embodiments, the calibration apparatus/method disclosed herein includes entire respin of the differential amplifier to reduce electromagnetic noise. In some embodiments, the calibration apparatus/method disclosed herein includes use of a differential signal generator. In some embodiments, the calibration apparatus/method disclosed herein includes improvements on the signal generation aspects to avoid clipping.

In some embodiments, the calibration method/approach includes creating an airtight box with a passive speaker connected to an oscilloscope. By coupling the transducer (e.g., a vibrating membrane) to this calibration box containing the passive speaker, the transducer is set in motion, and the movement creates pressure that would make an other speaker move, and transforms the air pressure difference back into an electrical signal that can be read, such as on an oscilloscope. In some embodiments, standardizing via the calibration method/approach disclosed herein includes comparing the systems and devices for pulmonary treatment with a sample group of previous versions.

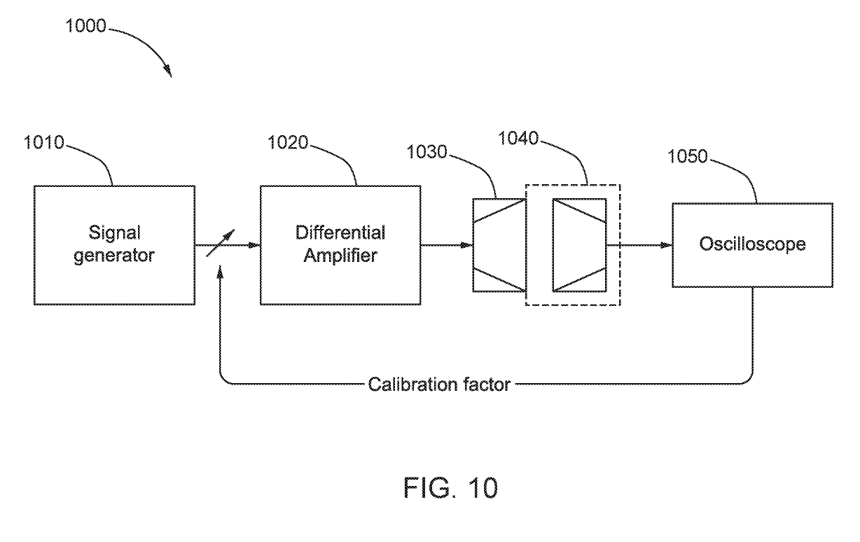

FIG. 10 illustrates a system 1000 for calibration and noise reduction for airway treatment, according to embodiments. The system 1000 includes a signal generator 1010 to generate electrical signals, which are then amplified by an amplifier 1020. A transducer 1030 is included in the system 1000 to convert the electrical signals into acoustic signals. The system 1000 also includes a calibration box 1040 including a passive speaker coupled to the transducer 1030. As described above, the acoustic signal (and the air pressure associated with the acoustic signal) generated by the transducer 1030 can excite the passive speaker in the calibration box 1040, which can transform the air pressure difference back into an electrical signal. An oscilloscope 1050 is connected to the calibration box 1040 to read the electrical signals generated by the calibration box and generate control signal that can facilitate calibration of the system 1000. In some embodiments, other measuring equipment such as a voltmeter can also be used to read the electrical signal generated by the calibration box.

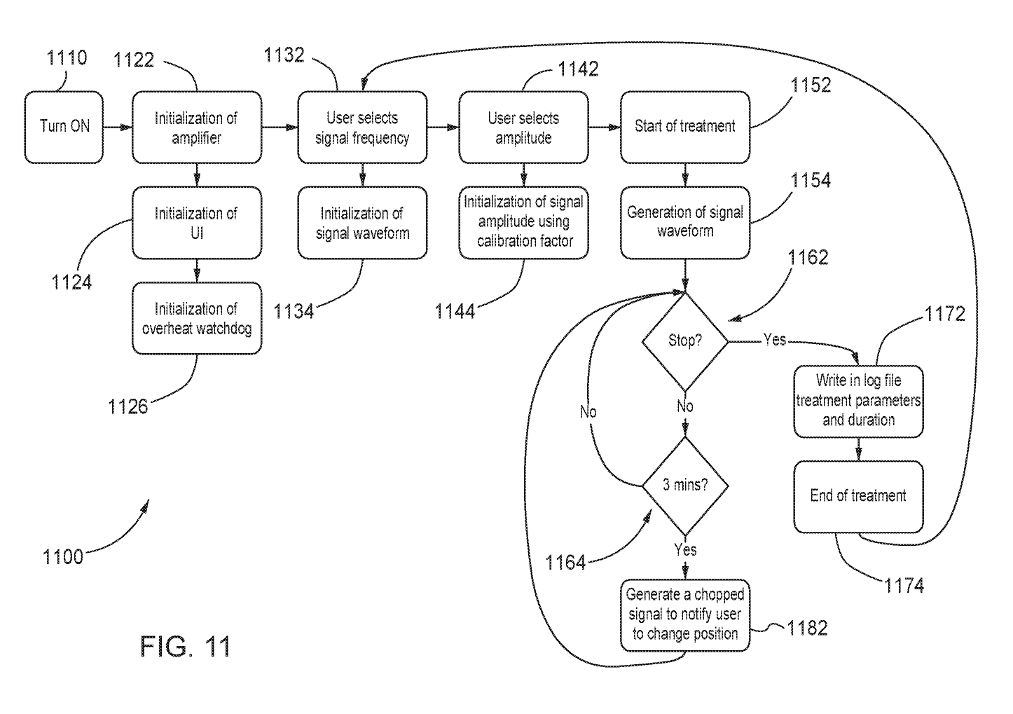

FIG. 11 illustrates a method 1100 for calibration and noise reduction for airway treatment, according to embodiments. At step 1110 of the method 1100, the system can be turned on. Then several initiation steps can be carried out, including initiation of the amplifier at step 1122, initiation of the user interface at step 1124, and initiation of overheat watchdog that can monitor the heat loading in the system, at step 1126. At step 1032, a user can select the signal frequency of electric signals generated by signal generators in the system. In addition, the signal waveform can be initialized at step 1134. For example, the desired waveform, such as sinusoidal wave or square wave, can also be set at this step.

At step 1142 in the method 1100, the user selects the desired amplitude of the acoustic signal. Alternatively, the user can selects the desired power level of the acoustic signal. At step 1144, the signal amplitude can be initialized using calibration factors such as default calibration factors, or calibration factors as used in most recent treatment.

The user starts the treatment at step 1152, followed by generation of signal waveform at step 1154. At step 1162, the user (or the system) determines whether to stop. In some embodiments, the user can decide to stop the treatment if a pause for the patient to cough-out the mucus is needed. In some embodiments, the user can decide to stop the treatment if it is the end of a predetermined treatment period. In some embodiments, the user can decide to stop the treatment for patient to change positions.

If the user decides to stop, the method 1100 proceeds to step 1172, where the treatment parameters, such as the frequency, amplitude, and duration, are written into a log file. The treatment can then ends at step 1174.

Alternatively, if at step 1162, the treatment does not stop. The method 1100 proceeds to step 1164, where the user or the system determines whether a predetermined amount of time (e.g., 3 minutes or any other amount of time that is appropriate) has lapsed since the beginning of the treatment. If so, the method proceeds to step 1182, where the system generates a chopped signal to notify the user to change position of the transducer. If not, the method 1100 moves back to step 1162.

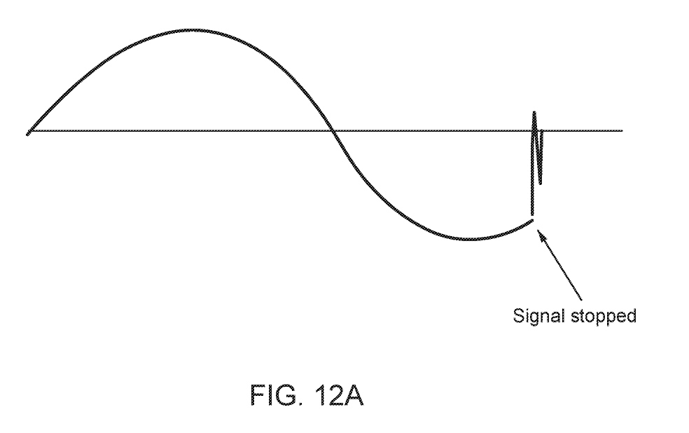

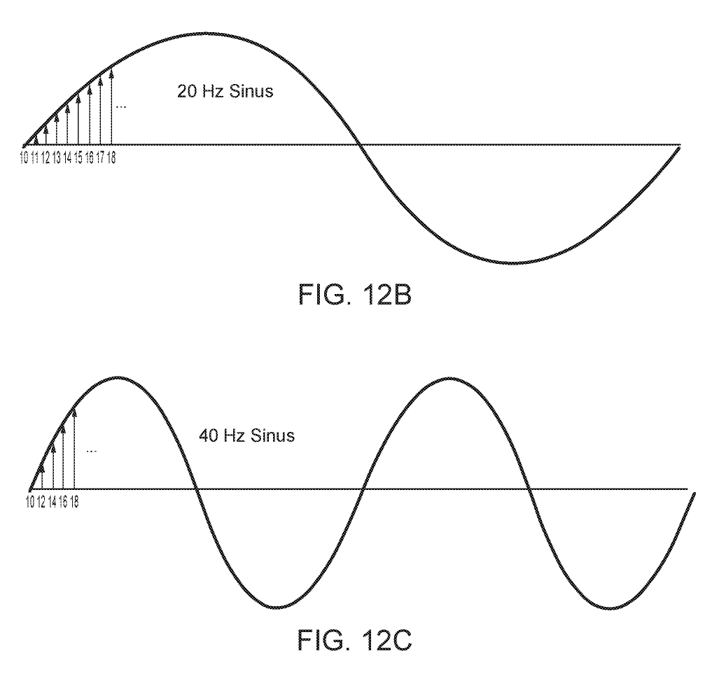

FIGS. 12A-12C show experimental signals acquired during calibration and noise reduction in airway treatments, according to embodiments. When the amplifier output is turned on or off, or when the frequency of the signal is changed by the operator, the airway treatment system/device can output a clipping sound that can negatively affect negatively the experience of the patient (see FIG. 12A). This clipping sound can occur since it is challenging for a sinusoidal signal curve to change from a non-zero value to zero instantaneously. Therefore, when the sound wave is created and amplified and the signal is generated by increasing and decreasing the amplified values in a sinus curve, it might happen that the signal is abruptly stopped at the time it is not perfectly at zero value. This is causing a generation of multiple harmonics to be generated that is heard as noise, the clipping sound.

To avoid this, the airway treatment systems, devices, and methods disclosed herein are directed to stopping the amplifier signal when it reached about zero amplitude. In such embodiments, there can be a small delay between the time the amplifier signal is stopped and the acoustic signal is heard. The maximum delay can be half a wavelength. For example, when the longest wavelength for the acoustic signal is about 20 Hz, it results in a 0.025 seconds delay, which is not perceptible by the human ear. This resulted in a better experience and smoother starts and stops.

In some embodiments, the same clipping sound can be heard when changing frequency while the acoustic signal is on. In some embodiments, when changing the frequency, a Portamento technique can be employed for compensation. In this manner, a smooth transition from one frequency to the next can be obtained. The Portamento technique is generally directed to gradually changing the frequency to make a smooth transition.

In some embodiments the airway treatment systems, devices, and methods disclosed herein account for the digital nature of the sinusoidal signal generation, i.e., the fact that the sinus generation is actually a sampled signal, when generated by a computer. In some embodiments, an acoustic sine wave at the lowest frequency that is desired (e.g., say 20 Hz) is generated, and then the offset of the sample in the signal can be changed to change the frequency. For example, if the frequency selected is 40 Hz, every next sample of the 20 Hz signal can be used. In this manner, a smooth transition of the output acoustic signal can be obtained (see FIGS. 12B and 12C).

In some cases, noise due to electromagnetic induction within the wiring of airway treatment systems and devices can be observed. For example, a touch screen cable can induce noise when the display was touched, and it can register at the vibrating membrane/transducer.

In some embodiments, the airway treatment systems and devices disclosed herein include shielded cables to minimize noise. In some embodiments, the airway treatment systems and devices disclosed herein include a low noise, class-D version for the differential amplifier.

In some embodiments, the airway treatment systems and devices disclosed herein include a differential signal between the signal generator and the amplifier. In this manner, by using 2 channels and subtracting them at the amplifier, ground noises can be eliminated by the subtraction of the signals.

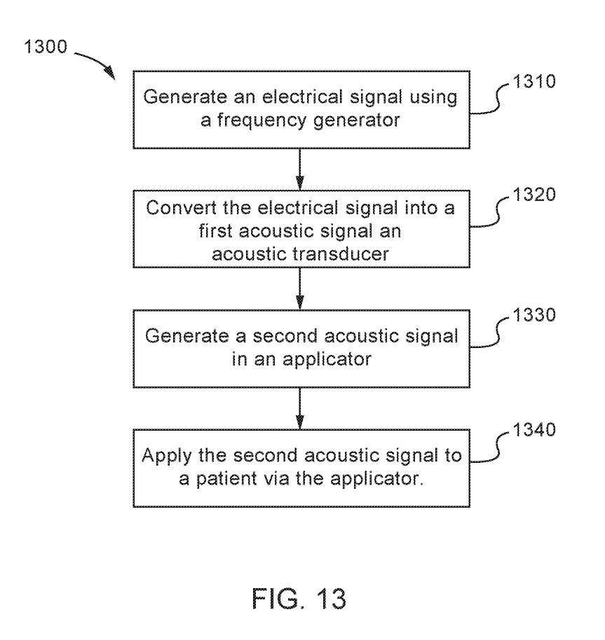

FIG. 13 illustrates a method 1300 of airway treatment, accordingly embodiments. The method 1300 includes generating an electrical signal using a frequency generator at step 1310. At step 1320, the electrical signal is converted into an acoustic signal using an acoustic transducer. The method 1300 also includes generating a second acoustic signal in an applicator based at least in part on the first acoustic signal, at step 1330. At step 1340, the second acoustic signal is applied to a patient via the applicator.

In some embodiments, the second acoustic signal is generated by exciting an applicator membrane in the applicator. In some embodiments, the applicator membrane includes silicone, or other pliable materials. In some embodiments, the applicator membrane is recessed from a contact interface between the applicator and the patient during operation. In this case, the applicator membrane can vibrate freely without touching the patient.

In some embodiments, the method 1300 further includes amplifying the second acoustic signal using an acoustic amplification chamber before applying the second acoustic signal to the patient. In some embodiments, the acoustic amplification chamber is made of polycarbonate or any other lightweight materials. In this manner, the applicator can be lightweight as well and allow convenient manipulation during treatment.

In some embodiments, the acoustic amplification chamber is filled with at least one of nitrogen or carbon dioxide so as to facilitate the transmission and/or amplification of acoustic signals. In some embodiments, the acoustic amplification chamber is filled with air.

In some embodiments, the method 1300 further includes transmitting the first acoustic signal from the acoustic transducer to the applicator through an acoustic waveguide. In some embodiments, the waveguide includes a flexible tube. In some embodiments, the flexible tube has a length greater than 2 feet (e.g., greater than 2 feet, greater than 3 feet, greater than 4 feet, or greater than 5 feet, including any values and sub ranges in between)

In some embodiments, the method 1300 further includes removing the applicator from the acoustic waveguide and coupling a second applicator to the acoustic waveguide.

In some embodiments, the method 1300 further includes reducing a cross sectional size of the first acoustic signal. This can be achieved by, for example, using an adapter that has a decreasing cross sectional area as the acoustic wave propagates within the adapter.

In some embodiments, reducing the cross sectional size of the first acoustic signal includes reducing the first cross sectional size by at least three fold (e.g., at least 3-fold, at least 5-fold, or at least 10-fold, including any values and sub ranges in between).

In some embodiments, applying the second acoustic signal to the patient includes implementing a treatment protocol to treat a pulmonary disorder of the patient. In some embodiments, applying the second acoustic signal to the patient includes implementing a treatment protocol to treat chronic obstructive pulmonary disease (COPD) of the patient. In some embodiments, applying the second acoustic signal to the patient includes implementing a treatment protocol to treat emphysema of the patient. In some embodiments, applying the second acoustic signal to the patient includes implementing a treatment protocol to treat lower efficiency respiratory function of the patient.

In some embodiments, the method 1300 further includes transmitting a portion of the first acoustic signal to a second applicator, generating a third acoustic signal in the second applicator based at least in part on the portion of the first acoustic signal, and applying the third acoustic signal to another patient via the second applicator. In this manner, multiple patients can be treated simultaneously using a common acoustic transducer (but different applicators).

The systems, device, and methods disclosed herein can be employed for airway treatment. In some embodiments, the systems, device, and methods disclosed herein are usable for treatment of pulmonary disorders such as, but not limited to, chronic obstructive pulmonary disease (COPD), emphysema, lower efficiency respiratory function, and/or the like.

Some embodiments described herein relate to a computer storage product with a non-transitory computer-readable medium (also referred to as a non-transitory processor-readable medium) having instructions or computer code thereon for performing various computer-implemented operations. The computer-readable medium (or processor-readable medium) is non-transitory in the sense that it does not include transitory propagating signals (e.g., a propagating electromagnetic wave carrying information on a transmission medium such as space or a cable). The media and computer code (also referred to herein as code) may be those designed and constructed for the specific purpose or purposes. Examples of non-transitory computer-readable media include, but are not limited to: flash memory, magnetic storage media such as hard disks, optical storage media such as Compact Disc/Digital Video Discs (CD/DVDs), Compact Disc-Read Only Memories (CD-ROMs), magneto-optical storage media such as optical disks, carrier wave signal processing modules, and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using Java, C++, or other programming languages and/or other development tools.

Where methods and/or schematics described above indicate certain events and/or flow patterns occurring in certain order, the ordering of certain events and/or flow patterns may be modified. Additionally certain events may be performed concurrently in parallel processes when possible, as well as performed sequentially.

1. An apparatus, comprising:
a signal generator configured to generate an electrical signal;
an acoustic transducer operably coupled to the signal generator, the acoustic transducer configured to convert the electrical signal into a first acoustic signal; and
an applicator operably coupled to the acoustic transducer, the applicator configured to receive the first acoustic signal from the acoustic transducer, the applicator including:
an acoustic generator configured to generate a second acoustic signal based on the first acoustic signal; and
an applicator interface configured to apply the second acoustic signal to a patient.

2. The apparatus of claim 1, wherein the applicator further includes an acoustic amplification chamber configured to amplify the first acoustic signal, the acoustic generator including an applicator membrane configured to seal the acoustic amplification chamber and to generate the second acoustic signal.

3. The apparatus of claim 2, wherein the material of the acoustic amplification chamber includes a polycarbonate, or silicone, or both.

4. The apparatus of claim 2, wherein the acoustic amplification chamber is filled with at least one of air, nitrogen gas, and carbon dioxide gas.

5. (canceled)

6. The apparatus of claim 2, wherein the applicator membrane is recessed from the applicator interface.

7. The apparatus of claim 1, further comprising:
a waveguide, disposed between the acoustic transducer and the applicator, the waveguide configured to transmit the first acoustic signal from the acoustic transducer to the applicator.

8. The apparatus of claim 6, wherein the waveguide includes a flexible tube having a length greater than about 2 feet.

9. (canceled)

10. The apparatus of claim 7, wherein the applicator is removably coupled to the waveguide.

11. The apparatus of claim 7, wherein a cross sectional area of the acoustic transducer is about 3 times to about 20 times greater than a cross sectional area of the waveguide.

12. (canceled)

13. (canceled)

14. The apparatus of claim 11, further comprising an adapter configured to couple the acoustic transducer to the waveguide, wherein the adapter includes:
a first end configured to be connected to the acoustic transducer;
a second end configured to be connected to the waveguide; and
a side wall portion disposed between the first end and the second end, the side wall portion being generally parabolic from the first end to the second end.

15. The apparatus of claim 11, further comprising an adapter configured to couple the acoustic transducer to the waveguide, wherein the adapter includes:
a first end configured to be connected to the acoustic transducer;
a second end configured to be connected to the waveguide; and
a side wall portion disposed between the first end and the second end, the side wall portion being generally hyperbolic from the first end to the second end.

16. The apparatus of claim 1, wherein the applicator is a first applicator, further comprising:
a second applicator operably coupled to the acoustic transducer, the second applicator configured to receive a portion of the first acoustic signal and generate a third acoustic signal based on the portion of the first acoustic signal, the second applicator including a second applicator interface configured to apply the third acoustic signal to another patient.

17. The apparatus of claim 1, further comprising:
a speaker operably coupled to the acoustic transducer, the speaker configured to receive a portion of the first acoustic signal; and
a measurement device operably coupled to the speaker, the measurement device configured to measure at least one parameter of the first acoustic signal.

18. The apparatus of claim 17, wherein the at least one parameter includes one or more of an amplitude of the first acoustic signal and a frequency of the first acoustic signal.

19.-22. (canceled)

23. A method, comprising:
generating an electrical signal using a signal generator;
converting the electrical signal into a first acoustic signal using an acoustic transducer;
generating a second acoustic signal in an applicator based at least in part on the first acoustic signal; and
applying the second acoustic signal to a patient via an applicator interface in the applicator.

24. The method of claim 23, wherein generating the second acoustic signal includes exciting an applicator membrane in the applicator.

25.-26. (canceled)

27. The method of claim 23, further comprising amplifying the second acoustic signal using an acoustic amplification chamber before applying the second acoustic signal to the patient.

28. (canceled)

29. (canceled)

30. The method of claim 23, further comprising:
transmitting the first acoustic signal from the acoustic transducer to the applicator through a waveguide.

31.-36. (canceled)

37. The method of claim 23, wherein applying the second acoustic signal to the patient includes implementing a treatment protocol to treat a pulmonary disorder of the patient.

38.-40. (canceled)

41. The method of claim 23, further comprising:
transmitting a portion of the first acoustic signal to a second applicator;
generating a third acoustic signal in the second applicator based at least in part on the portion of the first acoustic signal; and
applying the third acoustic signal to another patient via the second applicator.

42. The method of claim 23, further comprising:
receiving a portion of the first acoustic signal using a passive speaker;
measuring at least one parameter of the first acoustic signal; and
changing at least one operation parameter of the acoustic transducer based on the at least one parameter of the first acoustic signal.

43.-45. (canceled)

46. An apparatus for airway treatment, the apparatus comprising:
a signal generator configured to generate an electrical signal;
an acoustic transducer including a transducer membrane and operably coupled to the signal generator, the acoustic transducer configured to convert the electrical signal into a first acoustic signal;
a flexible acoustic waveguide operably coupled to the acoustic transducer, the acoustic waveguide configured to transmit the first acoustic signal; and
an applicator, operably coupled to the flexible acoustic waveguide, to receive the first acoustic signal, the applicator including:
an applicator membrane to generate a second acoustic signal based on the first acoustic signal;
an applicator interface to apply the second acoustic signal to a patient,
wherein the applicator membrane is recessed from the applicator interface.
